(19) United States
(12) Patent Application Publication
(10) Pub. No.: US 2012/0253058 A1
(43) Pub. Date: Oct. 4, 2012

(54) Manufacture of difluoroethylene carbonate, trifluoroethylene carbonate and tetrafluoroethylene carbonate

(75) Inventors: Jens Olschimke, Hannover (DE); Dirk Seffer, Hannover (DE); Martin Bomkamp, Hannover (DE)

(73) Assignee: SOLVAY FLUOR GMBH, Hannover (DE)

(21) Appl. No.: 13/497,316

(22) PCT Filed: Sep. 27, 2010
(86) PCT No.: PCT/EP2010/064221

§ 371 Date: Mar. 21, 2012

(30) Foreign Application Priority Data
Sep. 28, 2009 (EP) 09171491.5

(51) Int. Cl. C07C 68/06; H01L 21/306

(52) U.S. Cl. 558/260; 438/745; 257/E21.219

(57) ABSTRACT

Difluoroethylene carbonate, trifluoroethylene and tetrafluoroethylene carbonate are produced by the reaction between elemental fluorine and ethylene carbonate or fluorinated ethylene carbonates with a lower degree of fluorination.

### CROSS-REFERENCE TO RELATED APPLICATIONS

The present application is a U.S. national stage entry under 35 U.S.C. §371 of International Application No. PCT/EP2010/064221 filed Sep. 27, 2010, which claims benefit of European patent application number 09171491.5 filed on Sep. 28, 2009, the complete content of this application being incorporated herein by reference for all purposes.

### TECHNICAL FIELD OF THE INVENTION

The present invention concerns a process for the manufacture of difluoroethylene carbonate, trifluoroethylene carbonate and tetrafluoroethylene carbonate by reacting ethylene carbonate, fluoroethylene carbonate, 4,4-difluoroethylene carbonate, cis or trans 4,5-difluoroethylene carbonate or 4,4-difluoroethylene carbonate, and, for the manufacture of tetrafluoroethylene carbonate, also by reacting of trifluoroethylene carbonate, with elemental fluorine.

### BACKGROUND

Difluoroethylene carbonate, trifluoroethylene carbonate and tetrafluoroethylene carbonate are useful as solvents and additives for lithium ion batteries, and dielectric for capacitors. JP patent application 08-222485 mentions that difluoroethylene carbonate and tetrafluoroethylene carbonate are suitable as dielectric for capacitors and can be manufactured from ethylene carbonate by fluorination.

### SUMMARY OF THE PRESENT INVENTION

The process for the manufacture of difluoroethylene carbonate, trifluoroethylene carbonate and/or tetrafluoroethylene carbonate according to the present invention comprises a step
a) wherein a starting material with a lower degree of fluorination selected from the group consisting of ethylene carbonate, fluoroethylene carbonate, 4,4-difluoroethylene carbonate, cis-4,5-difluoroethylene carbonate, trans-4,5-difluoroethylene carbonate, or a mixture of two or more thereof is reacted in the liquid phase with elemental fluorine (F₂) to form trifluoroethylene carbonate,
b) wherein a starting material with a lower degree of fluorination selected from the group consisting of ethylene carbonate, fluoroethylene carbonate, 4,4-difluoroethylene carbonate, cis-4,5-difluoroethylene carbonate, trans-4,5-difluoroethylene carbonate, trifluoroethylene carbonate or a mixture of two or more thereof is reacted in the liquid phase with elemental fluorine (F₂) to form tetrafluoroethylene carbonate, or
c) wherein a starting material with a lower degree of fluorination selected from the group consisting of ethylene carbonate, fluoroethylene carbonate, or a mixture of two or more thereof is reacted in the liquid phase with elemental fluorine (F₂) to form difluoroethylene carbonate.

It was found that the fluorination of the indicated starting materials with elemental fluorine is a suitable way to manufacture difluoroethylene carbonate, trifluoroethylene carbonate and tetrafluoroethylene carbonate.

### DETAILED DESCRIPTION OF THE PREFERRED EMBODIMENTS

In a preferred embodiment, the manufacture of difluoroethylene carbonate, trifluoroethylene carbonate and tetrafluoroethylene carbonate comprises a step
d) wherein a starting material with a lower degree of fluorination selected from the group consisting of ethylene carbonate, fluoroethylene carbonate, 4,4-difluoroethylene carbonate, cis-4,5-difluoroethylene carbonate, trans-4,5-difluoroethylene carbonate, or a mixture of two or more thereof is reacted in the liquid phase with elemental fluorine (F₂) to form trifluoroethylene carbonate,
e) wherein a starting material with a lower degree of fluorination selected from the group consisting of ethylene carbonate, fluoroethylene carbonate, 4,4-difluoroethylene carbonate, cis-4,5-difluoroethylene carbonate, trans-4,5-difluoroethylene carbonate, trifluoroethylene carbonate or a mixture of two or more thereof is reacted in the liquid phase with elemental fluorine (F₂) to form tetrafluoroethylene carbonate,
f) wherein a starting material with a lower degree of fluorination selected from the group consisting of ethylene carbonate, fluoroethylene carbonate, or a mixture of two or more thereof is reacted in the liquid phase with elemental fluorine (F₂) to form difluoroethylene carbonate.
   and wherein the reaction is performed
   I) at a pressure higher than ambient pressure and/or
   II) with a condenser or a cooled trap or both in the off-gas line.

If performed according to this preferred embodiment, the yields are excellent.

Of course, if the process is directed to the manufacture of difluoroethylene carbonate or trifluoroethylene carbonate, always some higher fluorinated products such as tri- or tetrafluoroethylene carbonate are produced in a subsequent fluorination step.

According to one embodiment, the reaction is performed at a pressure higher than ambient pressure. This is the preferred embodiment and will be explained in detail later.

According to another embodiment, a condenser is located into the off gas line. By means of this condenser, a significant part or all of the tri- or tetrafluorinated carbonate which is entrained in the off gas, can be recovered. Often, and even usually, the elemental fluorine is introduced into the reaction mixture in diluted form. The preferred diluent is nitrogen, but other inert gases can be used also as diluent, e.g. the noble gases. While the fluorine reacts with the carbonate compound or compounds in the reaction mixture, the nitrogen (or any other inert gas) leaves the reactor via an off-line. The inventors have found that the gas stream which mainly consists of nitrogen, entrains some organic matter, especially the rather volatile difluoroethylene carbonate, trifluoroethylene carbonates (2 enantiomers) and tetrafluoroethylene carbonate. Since the off gas is usually treated in a washer or scrubber operated with water or acidic or basic aqueous solutions, and since difluoroethylene carbonate, trifluoroethylene carbonate and tetrafluoroethylene carbonate were identified to be susceptible to hydrolysis, according to one embodiment of the invention, a condenser is arranged in the off line for waste gas. It is preferred to locate the condenser on top or close to the top of the reactor so that condensed gas constituents flow back into the reaction mixture. The condenser can be operated with cooling water or cooling liquids. The temperature is regulated such that essentially all di-, tri- and tetrafluoroethylene carbonate is condensed and flows back to the reactor. The temperature of the condenser can be as low as technically possible. For example, cryomates can provide cooling liquids with a temperature down to about −100° C. The temperature of the cooling liquid is preferably in the range of −80° C. to 5° C.

Alternatively, one or more cooled traps is or are located in the off gas line. The contents of the trap contain predominantly di-, tri- and tetrafluoroethylene carbonate and some hydrogen fluoride. The contents can be separated by distillation to recover the respective pure organic carbonates. One or more traps cooled with liquid nitrogen can be applied. One has to be careful when warming up the content of the traps to avoid over-pressuring the system by nitrogen condensed in the traps. The temperature of the trap is preferably in the range of −80° C. to +5° C. Of course, if desired, several traps, 2, 3 or even 4 or more, can be arranged consecutively in the off gas line. Preferably, the traps downstream are kept at a lower temperature than the traps upstream. For example, traps can be consecutively arranged in the off gas line cooled to +5° C., −30° C. and −80° C.

It is also possible to combine the feature of performing the reaction at elevated pressure with the feature of using a condenser and/or using a trap. It is also possible to combine the features of using a condenser and a cooled trap.

In a preferred embodiment, the reaction is performed at a pressure higher than ambient pressure.

Of course, for the manufacture of trifluoroethylene carbonate, the starting material may contain also trifluoroethylene carbonate. That the starting material with a lower degree of fluorination is selected from the group consisting of said non-fluorinated or fluorinated carbonates does, of course, not exclude that compounds that do not react with fluorine, e.g. inert solvents as described below, are present in the reaction mixture, if desired.

The reaction can be performed batch-wise or continuously. For the selective manufacture of trifluoroethylene carbonate, the reaction can be performed in a cascade of reactors. This improves the selectivity of the process.

Preferably, the reaction is performed at a pressure higher than 1 bar (abs.). More preferably, the reaction is performed at a pressure equal to or higher than 2 bar (abs.). Especially preferably, the reaction is performed at a pressure equal to or higher than 3 bar (abs.).

Preferably, the reaction is performed at a pressure of equal to or lower than 20 bar (abs.). More preferably, the reaction is performed at a pressure equal to or lower than 15 bar (abs.). Especially preferably, the reaction is performed at a pressure equal to or lower than 12 bar (abs.). A preferred pressure range is from 4 to 8 bar (abs.), a more preferred pressure range from 5 to 7 bar (abs.).

It has to be noted that for each C—F bond which is formed during the reaction of a C—H bond and F₂, one molecule HF is formed. Thus, assuming a stoichiometric reaction between ethylene carbonate and fluorine (F₂), an F₂/H ratio of 4 is required, i.e. if 1 mol of ethylene carbonate is used as starting material, 4 moles of F₂ are stoichiometrically needed to achieve a complete fluorination of the ethylene carbonate. Thus, in the present invention, the ratio of F₂/H denotes the number of molecules of F₂ per H atom of the carbonate starting material which is to be substituted to form a C—F bond.

Generally, the reaction can be performed at a temperature equal to or higher than the melting point of the starting material to a temperature equal to or lower than 80° C. Preferred reaction temperatures are indicated below.

According to one embodiment, ethylene carbonate is reacted with elemental fluorine to produce difluoroethylene carbonate, trifluoroethylene carbonate or tetrafluoroethylene carbonate. The temperature at the beginning of the reaction can be equal or higher than 40° C. To improve selectivity and quantity of the desired products, the temperature of the reaction mixture can be decreased during progress of the reaction. The reaction is preferably performed at a temperature equal to or higher than 0° C. More preferably, it is performed at a temperature equal to or higher than 10° C. Preferably, the reaction is performed at a temperature equal to or lower than 50° C. More preferably, it is performed at a temperature equal to or lower than 45° C., and still more preferably, at a temperature equal to or lower than 35° C.

The F₂/H ratio is preferably equal to or greater than 3 if it is intended to produce trifluoroethylene carbonate from ethylene carbonate. It is preferably equal to or lower than 4.

If tetrafluoroethylene carbonate is to be produced by the reaction between ethylene carbonate and F₂, the F₂/H ratio is preferably equal to or greater than 4. It is preferably equal to or lower than 6.

According to a second embodiment, fluoroethylene carbonate is reacted with elemental fluorine to form difluoroethylene carbonate, trifluoroethylene carbonate or tetrafluoroethylene carbonate. The temperature at the beginning of the reaction can be equal or higher than 25° C. To improve selectivity and quantity of the desired products, the temperature of the reaction mixture can be decreased during progress of the reaction. The reaction is preferably performed at a temperature equal to or higher than 0° C. More preferably, it is performed at a temperature equal to or higher than 10° C. Preferably, it is performed at a temperature equal to or lower than 50° C. More preferably, it is performed at a temperature equal to or lower than 45° C., still more preferably, equal to or lower than 35° C.

The F₂/H ratio is preferably equal to or greater than 2 if it is intended to produce trifluoroethylene carbonate from fluoroethylene carbonate. It is preferably equal to or lower than 4. If, in this embodiment, tetrafluoroethylene carbonate is to be produced, the F₂/H ratio is preferably equal to or greater than 3. It is preferably equal to or lower than 5.

According to a third embodiment, 4,4-difluoroethylene carbonate, 4,5-difluoroethylene carbonate (cis isomer, trans isomer or cis and trans isomer) or a mixture thereof is reacted with elemental fluorine. The reaction is preferably performed at a temperature equal to or higher than the melting point of the mixture. More preferably, it is performed at a temperature equal to or higher than 0° C. Preferably, it is performed at a temperature equal to or lower than 50° C. More preferably, it is performed at a temperature equal to or lower than 45° C., still more preferably equal to or lower than 35° C.

The F₂/H ratio is preferably equal to or greater than 1 if trifluoroethylene carbonate is to be produced from any of the difluoroethylene carbonates. It is preferably equal to or lower than 3. If tetrafluoroethylene carbonate is to be produced, the F₂/H ratio is preferably equal to or greater than 2. It is preferably equal to or lower than 4.

According to a fourth embodiment, a starting material mixture is used which contains two or more of ethylene carbonate, fluoroethylene carbonate, 4,4-difluoroethylene carbonate, cis-4,5-difluoroethylene carbonate, trans-4,5-difluoroethylene carbonate, and trifluoroethylene carbonate. If trifluoroethylene carbonate is to be produced, trifluoroethylene carbonate may be present, but it is preferred if this compound is absent or present only in minor amounts to reduce the degree of a further reaction to tetrafluoroethylene carbonate. For each C—H bond which is to be substituted by a C—F bond, preferably 1 to 1.5 molecules of F₂ are applied.

Mixtures containing ethylene carbonate, fluoroethylene carbonate and the isomers of difluoroethylene carbonate, which can be used as starting material, are for example those mixtures which are low boiler distillates, high boiler distillates or high boiler distillation residues obtained in a process for the manufacture of lower fluorinated ethylene carbonates. Elemental fluorine (F₂) can be applied in neat form, if desired. In this case, the reaction temperature is preferably kept in the lower region of the range given above due to the high heat release by the fluorination reaction.

Preferably, fluorine is introduced in diluted form into the reaction. The preferred diluent gas is nitrogen, but, if desired, noble gases, e.g. helium and/or argon can be applied as diluent gas or gases or as additional diluent gases. While any volume ratio of F₂ and inert gas in the range of 1:99 to 99:1 is suitable, a concentration of 2 to 50% by volume of fluorine in the mixture of fluorine and inert gas or inert gasses is very suitable. A mixture of elemental fluorine and nitrogen is preferred. The concentration of fluorine is greater than 0% by volume. It is preferably equal to or greater than 5% by volume. It is more preferably equal to or greater than 12% by volume. The concentration of fluorine is preferably equal to or lower than 25% by volume. Preferably, it is equal to or lower than 18% by volume.

The reaction temperature and the pressure given above can be varied during the reaction. For example, when ethylene carbonate, monofluoroethylene carbonate or their mixtures are used as starting material, the reaction temperature is selected preferably in the lower region of the given range because of the higher reactivity of carbonates with a lower degree of fluorination.

It is possible to control the reaction between fluorine and the starting material so that the production of trifluoroethylene carbonate is favored. Here, the molar ratio between fluorine and the starting material is selected such that it is not greater than the stoichiometric amount needed to convert all ethylene carbonate or fluorosubstituted ethylene carbonate to trifluoroethylene carbonate; further, the pressure may be kept in the lower range, e.g. from greater than 1 to about 6 bar (abs). This allows a part of the trifluoroethylene carbonate to evaporate from the reaction mixture and thus to avoid being further fluorinated.

If it is intended to produce predominantly tetrafluoroethylene carbonate, the molar ratio between fluorine and the starting material is such that the substitution of all C—H bonds by C—F bond is possible. Further, the pressure may be kept in the upper range, e.g. in the range of 5 to 12 bar (abs.) because this prevents too much trifluoroethylene carbonate to evaporate and thus, to avoid being perfluorinated.

If desired, the reaction between the starting material and fluorine is performed in a presence of a solvent. Suitable solvents are those solvents which do not react with fluorine to form undesired by-products. Linear or cyclic perfluorocarbons, for example, fluorinated ethers sold by Solvay Solexis under the tradenames Galden® and Fomblin®, tetrafluoroethylene carbonate or hydrogen fluoride can be applied as solvents.

In a preferred embodiment, the reaction is performed in the absence of a solvent. Thus, the reaction is preferably performed using neat, undiluted carbonate.

A good mixing of starting material and fluorine gas or mixture of fluorine gas and inert gas is advantageous. For example, the preferred F₂/N₂ mixture is introduced into the reaction mixture by means of a frit allows a good distribution of small gas bubbles into the liquid starting material or reaction mixture. Alternatively, the fluorine gas or fluorine gas containing gas mixture can be contacted with the starting material or reaction mixture as described in U.S. Pat. No. 7,268,238. The reaction mixture is circulated and the contact between the liquid and gaseous reactants is improved by packings inside the reactor.

The workup can be performed by contacting the reaction mixture with water to remove any HF and other water-soluble impurities. The resultant pre-purified reaction mixture is then, optionally after drying, e.g. over salts applied for this purpose, e.g. magnesium sulfate, and then distilled to obtain the desired pure difluoroethylene carbonate, trifluoroethylene carbonate or tetrafluoroethylene carbonate. It has to be noted that difluoroethylene carbonate, trifluoroethylene carbonate and tetrafluoroethylene carbonate are sensitive to hydrolysis.

Alternatively, the reaction mixture is distilled. Optionally, HF is removed before distillation, e.g. by stripping the reaction mixture with an inert gas. Nitrogen is very suitable as stripping gas; but argon or helium or their mixtures with nitrogen can be used as well. Small amounts of HF can be removed by absorption with NaF or KF.

The method for the manufacture of difluoroethylene carbonate, trifluoroethylene carbonate and tetrafluoroethylene carbonate is preferably performed such that formed difluoroethylene carbonate, trifluoroethylene carbonate and tetrafluoroethylene carbonate do not come into contact with glass and Lewis acids, especially Lewis acids which are present in glass or which are formed from constituents of glass in contact with HF.

Glass or ceramics contain Si—O bonds. Difluoroethylene carbonate, trifluoroethylene carbonate and tetrafluoroethylene carbonate are sensitive towards hydrolysis. Glass and ceramics with Si—O bonds are often sensitive to hydrogen fluoride because HF reacts under the formation of water and SiF₄. Water, as mentioned above, causes hydrolysis of tri- and tetrafluoroethylene carbonates. Since probably a very minute amount of water or HF adhering to the glass items or in the fluorinated carbonate cannot be excluded a reaction as described above may take place. The Lewis acids or Lewis acid precursors contained in glass are set free and react with HF. For example, aluminium oxide is contained in many glasses and forms Al—F bonds when contacted with HF. The resulting components are Lewis acids and are considered to accelerate the decomposition of tri- and tetrafluoroethylene carbonates. It also assumed that the contact of tri- and tetrafluoroethylene carbonate with metals which contain Lewis acid precursors should be avoided.

Accordingly, it is preferred to perform the process of the present invention not in apparatus which contain glass parts, ceramic parts or metal or metal alloy parts which contain Lewis acid precursors (e.g., aluminium or aluminium containing alloys) and are not resistant to HF and which could or would come into contact with the tri- or tetrafluoroethylene carbonate. It is preferred to perform the process of the invention in apparatus containing only parts made of HF-resistant metals or polymeric material. Parts made from stainless steel, HF-resistant alloys like Inconel or Hastelloy are preferred, Suitable polymers are, for example, partially or perfluorinated polymers, as well as polyalkylene polymers and other types of polymers. For example, PFA (perfluoroalkoxyalkane co-polymer), PTFE (polytetrafluoroethylene), PE (polyethylene), or PVDF (polyvinylidene difluoride) are very suitable. The suitability of other polymers can easily be checked. Preferably, the reactor, pipes, stripping units, distillation towers, storage tanks, and other items which come into contact with difluoroethylene carbonate, trifluoroethylene carbonate and tetrafluoroethylene carbonate are made of stainless steel, Inconel, Hastelloy or other resistant material, or of said polymeric material, or lined with it. The term “resistant” denotes materials which do not react with difluoroethylene carbonate, trifluoroethylene carbonate and tetrafluoroethylene carbonate in an undesired way.

As described above, difluoroethylene carbonate, trifluoroethylene carbonate and tetrafluoroethylene carbonate are contacted during their manufacture preferably only with parts which do not cause the decomposition of these compounds. In another embodiment, difluoroethylene carbonate, trifluoroethylene carbonate and tetrafluoroethylene carbonate are handled in this way not only during their manufacture, but from the moment of their manufacture until they are applied, e.g. as battery solvent, including storage, packaging, transport, additional purification steps, mixing with other components of electrolyte mixtures or electrolyte solutions, e.g. their mixture with ethylene carbonate, propylene carbonate, optionally also including Li salt, e.g. LiPF₆.

The term “handling” denotes any step of life cycle of the compounds starting from the moment they come into existence by manufacture to the moment when they have lost any technical interest for use, i.e. when they are no longer applied, but ready for destruction, dumping or have otherwise become technically useless. The term “handling” especially includes the manufacture of the compounds, the storage of the compounds, and any step during which they are used. The term “handling” includes passing the carbonates during their manufacture or use through pipes, valves, mixing apparatus, filling them, or mixtures containing them, into battery housings etc.

The process of the invention allows the manufacture of difluoroethylene carbonate, trifluoroethylene carbonate and tetrafluoroethylene carbonate in an easy and reliable manner. In preferred embodiments, the selective manufacture of difluoroethylene carbonate, the selective manufacture of trifluoroethylene carbonate and the selective manufacture of tetrafluoroethylene carbonate are possible.

The difluoroethylene carbonate, trifluoroethylene carbonate and tetrafluoroethylene carbonate can be applied as additive for lithium ion batteries. It was found that they are also useful as etching gas for the manufacture of semiconductors, flat panel displays and solar panels. They have no impact on the ozone layer and their GWP is estimated to be quite low because they tend to hydrolyse in the presence of water. They can, for example, be applied in an analogous manner as described in unpublished PCT patent application PCT/EP2009/058996 the content of which is incorporated herein entirely. They are usually applied in a plasma apparatus at relatively low pressures. Optionally, they are diluted with nitrogen, helium, argon, xenon or other additive or diluent gases). Helium and especially nitrogen are predominantly diluent gases. Argon and xenon are additive gases which dilute the fluorinated unsaturated C4 compound or compounds, but which also can influence the selectivity of the etching process.

Often, the pressure in the plasma chamber is equal to or below 150 Pa. Preferably, the pressure is from 1 to 120 Pa.

The use of these compounds in a process for the etching of items in the manufacture of semiconductors, flat panel displays and TFTs is another object of the present invention.

Difluoroethylene carbonate, trifluoroethylene carbonate and tetrafluoroethylene carbonate are also suitable as solvent or building block in synthesis, refrigerant, or flame retardant.

Should the disclosure of any patents, patent applications, and publications which are incorporated herein by reference conflict with the description of the present application to the extent that it may render a term unclear, the present description shall take precedence.

### EXAMPLES

The invention will now be described in examples without the intention to limit it thereto.

### General Procedure:

A reactor with gas inlets and gas outlet was applied. The starting material was ethylene carbonate. Fluorine was introduced in the form of a mixture consisting of 10 vol.-% fluorine and 90 vol-% nitrogen through a metal diffuser. Additionally, nitrogen was introduced separately into the reactor. The temperature of the reaction mixture was kept in a range of ± about 7° C. from the indicated average temperature, except in example 4 where the minimum temperature was 20.7° C. The composition of the reaction mixture was regularly determined by gas chromatography. The data are compiled in the following table 1.

TABLE 1
Reaction data

| | Example 1 | Example 2 | Example 3 | Example 4 | Example 5 | Example 6 |
|---|---|---|---|---|---|---|
| Starting product [g] | 2820 | 2860 | 2900 | 2760 | 2727 | 2749 |
| Raw yield [g] | 1406 | 2299 | 2891 | 2761 | 2760 | 3840 |
| Temperature*liquid | 41.9 | 37.1 | 36.1 | 35.3 | 23.9 | 20.8 |
| [° C.] | | | | | | |
| Temperature gas | 38.9/ | 36.9 | 36.5 | 36.4 | 31.2 | 25.8 |
| phase [° C.]* | | | | | | |
| Pressure diffuser | 2.64 | 3.64 | 5.07 | 5.97 | 0.17 | 2.6 |
| [bar]* | | | | | | |
| Pressure in reactor | 2.5 | 3.5 | 4.93 | 5.83 | 0.02 | 2.5 |
| [bar]* | | | | | | |
| N₂ flow [l/h]* | 20.6 | 18.7 | 11 | 10 | 39.5 | 20.9 |
| F₂/N₂ flow [l/h]* | 63 | 55.3 | 37.3 | 29.5 | 138.3 | 70.7 |
| Total volume F₂/N₂ | 20.900 | 20.200 | 20.000 | 21.540 | 17.000 | 16.950 |
| [l] | | | | | | |

*Average value

Regularly, after specific volumes of added F₂/N₂, the composition of the liquid phase (reaction mixture) in the reactor was analyzed by gas chromatography.

Selected results are given in tables 2 to 7.

TABLE 2
Analytical data of example 1 (GC-%):

| | | | | | | | |
|---|---|---|---|---|---|---|---|
| F₄EC | 0 | 0 | 0 | 0.06 | 0.2 | 0.8 | 2.44 |
| F₃EC | 0 | 0.9 | 2.9 | 5.9 | 16.5 | 34.7 | 69.5 |
| Trans- | 0 | 4.1 | 12.5 | 27.6 | 44.4 | 44.8 | 24.4 |
| F₂EC | | | | | | | |
| 4,4- | 0 | 1 | 2.9 | 5.7 | 7.1 | 3.8 | 0.3 |
| F₂EC | | | | | | | |
| Cis- | 0 | 1.85 | 5.8 | 12.6 | 17.9 | 14.7 | 3.3 |
| F₂EC | | | | | | | |
| F₁EC | 0 | 38.5 | 58.4 | 46.9 | 13.8 | 1.3 | 0.05 |
| EC | 100 | 53.7 | 17.7 | 1.2 | 0 | 0 | 0 |
| Volume | 0 | 4.800 | 8.800 | 12.000 | 15.250 | 17.650 | 20.950 |
| of | | | | | | | |
| F₂/N₂ | | | | | | | |
| added | | | | | | | |
| [l] | | | | | | | |

TABLE 3
Analytical data of example 2 (GC-%):

| | | | | | | | |
|---|---|---|---|---|---|---|---|
| F₄EC | 0 | 0 | 0 | 0 | 0 | 0.5 | 4.2 |
| F₃EC | 0 | 2.1 | 2.9 | 4.2 | 9.1 | 22 | 65.3 |
| Trans- | 0 | 3.3 | 6.4 | 15.5 | 33.7 | 46.1 | 25.7 |
| F₂EC | | | | | | | |
| 4,4- | 0 | 0.5 | 1.3 | 3.4 | 6.6 | 6.5 | 0.5 |
| F₂EC | | | | | | | |
| Cis- | 0 | 1.1 | 2.4 | 6.6 | 13.2 | 17.3 | 4.3 |
| F₂EC | | | | | | | |
| F₁EC | 0 | 24.6 | 40.4 | 57.1 | 36.6 | 7.6 | 0.02 |
| EC | 100 | 68.3 | 46.6 | 13.2 | 0.7 | 0.02 | 0 |
| Volume | 0 | 2.700 | 4.900 | 9.100 | 12.800 | 16.400 | 20.800 |
| of | | | | | | | |
| F₂/N₂ | | | | | | | |
| added | | | | | | | |
| [l] | | | | | | | |

TABLE 4
Analytical data of example 3 (GC-%):

| | | | | | | | |
|---|---|---|---|---|---|---|---|
| F₄EC | 0 | 0 | 0 | 0.13 | 0.5 | 1.7 | 2.9 |
| F₃EC | 0 | 2.3 | 5 | 11.1 | 16.6 | 38.6 | 56.1 |
| Trans- | 0 | 5.1 | 15.8 | 35 | 42.5 | 41.9 | 33.1 |
| F₂EC | | | | | | | |
| 4,4- | 0 | 1.0 | 4.3 | 6.9 | 7.2 | 4 | 1.3 |
| F₂EC | | | | | | | |
| Cis- | 0 | 1.8 | 6.7 | 13.3 | 15.3 | 12.5 | 6.6 |
| F₂EC | | | | | | | |
| F₁EC | 0 | 36.0 | 56.6 | 33.2 | 18 | 1.3 | 0 |
| EC | 100 | 53.7 | 11.6 | 0.4 | 0 | 0 | 0 |
| Volume | 0 | 4.800 | 10.100 | 13.500 | 14.800 | 18.000 | 20.000 |
| of | | | | | | | |
| F₂/N₂ | | | | | | | |
| added | | | | | | | |
| [l] | | | | | | | |

TABLE 5
Analytical data of example 4 (GC-%):

| | | | | | | | |
|---|---|---|---|---|---|---|---|
| F₄EC | 0 | 0.04 | 0 | 0.04 | 0 | 2 | 9.7 |
| F₃EC | 0 | 2.5 | 5.9 | 10.2 | 19.4 | 48.6 | 75.4 |
| Trans- | 0 | 6.1 | 18.4 | 31.8 | 45.2 | 38.6 | 13.6 |
| F₂EC | | | | | | | |
| 4,4- | 0 | 1.2 | 3.8 | 6.2 | 7.5 | 2.7 | 0 |
| F₂EC | | | | | | | |
| Cis- | 0 | 2.3 | 7.5 | 12 | 15 | 7.9 | 1 |
| F₂EC | | | | | | | |
| F₁EC | 0 | 41 | 58.9 | 39.3 | 12.9 | 0.13 | 0.4 |
| EC | 100 | 46.9 | 7.6 | 0.3 | 0 | 0.03 | 0 |
| Volume | 0 | 5.500 | 10.100 | 12.300 | 14.840 | 18.340 | 21.540 |
| of | | | | | | | |
| F₂/N₂ | | | | | | | |
| added | | | | | | | |
| [l] | | | | | | | |

TABLE 6
Analytical data of example 5 (GC-%)

| | | | | | | | |
|---|---|---|---|---|---|---|---|
| F₃EC | 0 | 0.3 | 0.5 | 1.0 | 1.2 | 2.7 | 4.7 |
| Trans- | 0 | 3.7 | 5.3 | 12.2 | 13.0 | 29.2 | 37.7 |
| F₂EC | | | | | | | |
| 4,4- | 0 | 0.9 | 1.3 | 2.7 | 3.1 | 6.3 | 7.2 |
| F₂EC | | | | | | | |
| Cis- | 0 | 2.2 | 3.1 | 6.2 | 7.9 | 16.3 | 22.9 |
| F₂EC | | | | | | | |
| F₁EC | 0 | 42.6 | 50.9 | 62.6 | 63.8 | 44.7 | 27.5 |
| EC | 100 | 50.3 | 38.9 | 15.43 | 11.0 | 0.7 | 0.1 |
| Volume | 0 | 4.000 | 5.700 | 8.000 | 9.300 | 14.000 | 17.000 |
| of | | | | | | | |
| F₂/N₂ | | | | | | | |
| added | | | | | | | |
| [l] | | | | | | | |

TABLE 7
Analytical data of example 6 (GC-%)

| | | | | | | | |
|---|---|---|---|---|---|---|---|
| F₃EC | 0 | 0.6 | 1.0 | 1.4 | 2.0 | 5.2 | 13.6 |
| Trans- | 0 | 4.3 | 7.5 | 11.8 | 16.3 | 33.2 | 47.0 |
| F₂EC | | | | | | | |
| 4,4- | 0 | 1.1 | 1.9 | 2.9 | 3.8 | 6.7 | 7.3 |
| F₂EC | | | | | | | |
| Cis- | 0 | 2.3 | 4.1 | 6.3 | 8.5 | 15.9 | 20.6 |
| F₂EC | | | | | | | |
| F₁EC | 0 | 42.9 | 56.0 | 62.7 | 62.7 | 38.9 | 11.5 |
| EC | 100 | 48.8 | 29.5 | 14.9 | 6.7 | 0.04 | 0.03 |
| Volume | 0 | 4.050 | 5.950 | 7.750 | 9.050 | 12.750 | 16.500 |
| of | | | | | | | |
| F₂/N₂ | | | | | | | |
| added | | | | | | | |
| [l] | | | | | | | |

The respective raw products could then be separated by distillation. Tables 1, 6 and 7 show that the yield is much higher if the reaction is performed under pressure. As indicated by the results of examples 5 and 6, the reactor pressures of which are 0.02 bar and 2.5 bar, respectively, the amount of trifluoroethylene(F3EC) in example 5 (i.e., 4.7%), is much lower than that in example 6 (i.e., 13.6%). Further, the total amount of trans-F₂EC, 4,4-F₂EC, and cis-F₂EC in example 5 (i.e., 67.8%) is lower than that in example 6 (i.e., 74.9%). Those results clearly indicate that use of a pressure higher than ambient pressure leads to higher yield toward di-, tri- or tetrafluoroethylene, and can also avoid the loss of volatile products as of the above products.

### Example 7

### Storage of Trifluoroethylene Carbonate in a Glass Bottle

Trifluoroethylene carbonate was stored in a glass bottle. It was observed that pressure built up. This indicates a decomposition of the compound. In the gas space, SiF₄ was determined. This indicates a reaction of SiO₂ from the glass of the bottle with HF under formation of water and SiF₄.

### Example 8

### Storage of Trifluoroethylene Carbonate in an Aluminium Vessel

Trifluoroethylene carbonate is stored in an aluminium vessel. Pressure formation is observed indicating a decomposition of the stored product.

### Example 9

### Storage of Tetrafluoroethylene Carbonate in a Pressure Resistant Glass Bottle

Trifluoroethylene carbonate is stored in a pressure resistant glass bottle. It is observed that pressure builds up. This indicates a decomposition of the compound. In the gas space, SiF₄ was determined. This indicates a reaction of SiO₂ from the glass of the bottle with HF under formation of water and SiF₄.

1. A process for the manufacture of difluoroethylene carbonate, trifluoroethylene carbonate and/or tetrafluoroethylene carbonate which comprises a reaction step
a) wherein a starting material with a lower degree of fluorination selected from the group consisting of ethylene carbonate, fluoroethylene carbonate, 4,4-difluoroethylene carbonate, cis-4,5-difluoroethylene carbonate, trans-difluoroethylene carbonate, and a mixture of two or more thereof is reacted in a liquid phase with elemental fluorine (F₂) to form trifluoroethylene carbonate, or
b) wherein a starting material with a lower degree of fluorination selected from the group consisting of ethylene carbonate, fluoroethylene carbonate, 4,4-difluoroethylene carbonate, cis-4,5-difluoroethylene carbonate, trans-difluoroethylene carbonate, trifluoroethylene carbonate, and a mixture of two or more thereof is reacted in a liquid phase with elemental fluorine (F₂) to form tetrafluoroethylene carbonate, or
c) wherein a starting material with a lower degree of fluorination selected from the group consisting of ethylene carbonate, fluoroethylene carbonate, and a mixture of two or more thereof is reacted in a liquid phase with elemental fluorine (F₂) to form difluoroethylene carbonate.

2. The process of claim 1, wherein the reaction between the starting material and elemental fluorine is performed
I) at a pressure higher than ambient pressure and/or
II) with a condenser or a cooled trap or both in an off-gas line.

3. The process of claim 2 wherein the reaction between the starting material and elemental fluorine is performed at a pressure equal to or higher than 3 bar (abs.).

4. The process of claim 2 wherein the reaction between the starting material and elemental fluorine is performed at a pressure equal to or lower than 12 bar (abs.).

5. The process of claim 1 wherein ethylene carbonate or fluoroethylene carbonate is used as starting material.

6. The process of claim 1 wherein a difluoroethylene carbonate selected from the group consisting of 4,4-difluoroethylene carbonate, cis-4,5-difluoroethylene carbonate, trans-4,5-difluoroethylene carbonate, and any mixture thereof is used as starting material.

7. The process of claim 1 wherein a mixture containing at least two compounds selected from the group consisting of ethylene carbonate, fluoroethylene carbonate, 4,4-difluoroethylene carbonate, cis-4,5-difluoroethylene carbonate, and trans-4,5-difluoroethylene carbonate is used as starting material.

8. The process of claim 1 wherein the formed difluoroethylene carbonate, trifluoroethylene carbonate and tetrafluoroethylene carbonate are isolated and stored.

9. The process according to claim 1 wherein the formed difluoroethylene carbonate, trifluoroethylene carbonate and tetrafluoroethylene carbonate are contacted only with HF-resistant material.

10. The process of claim 9 wherein the HF-resistant material is stainless steel or an organic polymeric material.

11. A method for the manufacture of semiconductors, flat panel displays and solar panels comprising a step of etching wherein difluoroethylene carbonate, trifluoroethylene carbonate and tetrafluoroethylene carbonate are used as etching agent.

12. A method of handling difluoroethylene carbonate, trifluoroethylene carbonate and tetrafluoroethylene carbonate wherein difluoroethylene carbonate, trifluoroethylene carbonate and tetrafluoroethylene carbonate are not contacted with Lewis acids or Lewis acid precursors.

13. The method of claim 12 wherein difluoroethylene carbonate, trifluoroethylene carbonate and tetrafluoroethylene carbonate are not contacted with glass, ceramics, or aluminium parts containing aluminium alloys.

14. The method of claim 12 wherein difluoroethylene carbonate, trifluoroethylene carbonate and tetrafluoroethylene carbonate is contacted with stainless steel, an HF-resistant alloy, or a polymeric material.

15. The process of claim 14 wherein the polymeric material is perfluorinated.

16. A method for refrigeration, synthesis, or flame retardation, comprising using difluoroethylene carbonate, trifluoroethylene carbonate and tetrafluoroethylene carbonate as solvent or building block in synthesis, as refrigerant, or as flame retardant.
